(19) United States
(12) Patent Application Publication
(10) Pub. No.: US 2025/0009518 A1
(43) Pub. Date: Jan. 9, 2025

(54) A TISSUE REGENERATION SCAFFOLD

(71) Applicant: THE PROVOST, FELLOWS, SCHOLARS AND OTHER MEMBERS OF BOARD OF TRINITY COLLEGE DUBLIN, Dublin (IE)

(72) Inventors: Daniel KELLY, Dublin (IE); Klan EICHHOLZ, Dublin (IE); Orquidea GARCIA, Irvine, CA (US)

(73) Assignee: THE PROVOST, FELLOWS, SCHOLARS AND OTHER MEMBERS OF BOARD OF TRINITY COLLEGE DUBLIN, Dublin (IE)

(21) Appl. No.: 18/709,946

(22) PCT Filed: Nov. 17, 2022
(86) PCT No.: PCT/EP2022/082356

§ 371 Date: May 14, 2024

Related U.S. Application Data
(60) Provisional application No. 63/280,443, filed on Nov. 17, 2021.

(51) Int. Cl. A61F 2/30; A61F 2/28

(52) U.S. Cl. CPC A61F 2/30771; A61F 2/2846; A61F 2/30756; A61F 2002/30235; A61F 2002/30766; A61F 2002/3092; A61F 2310/00796; A61F 2310/00976

(57) ABSTRACT

A melt electrowritten tissue regeneration scaffold (1, 10, 20, 31, 41) comprises a first section (1, 10, 21, 31, 43) comprising a plurality of first printed layers (6), each first printed layer comprising one or more printed fibres (7, 8) having a diameter of less than 40 μm arranged in a lattice and defining a plurality of openings (9) having a diameter of less than 800 pm. At least some of the first printed layers are arranged such that the openings are aligned to define 3-D pores (5) that extend at least partially through the first section. The scaffold has a specific surface area of at least 150 mm²/mm³.

### FIELD OF THE INVENTION

The present invention relates to a tissue regeneration scaffold and in particular a bone and osteochondral regeneration and repair scaffold. Also contemplated is the use of the tissue regeneration scaffold to repair hard tissue such as bone or cartilage in a mammal.

### BACKGROUND TO THE INVENTION

There are a host of scaffolds which have been developed for bone tissue engineering applications. Specific to this application, there are several 3D printed scaffolds which have been developed [Bose et al (2013) Bone tissue engineering using 3D printing, materials today, 16(2): 496-504], where the general consensus has typically been that larger pores allow greater vascularization and bone formation. A recent review of the literature by experts in the field has found that the reality is likely more complicated than this, demonstrating that there is a fine balance in designing a scaffold architecture which is osteoinductive, namely, that it drives stem cell osteogenic differentiation, which in turn promotes bone healing [Bohner & Miron (2020) A proposed mechanism for material induced heterotopic ossification, Materials Today, 22: 132-141]. It is proposed that porosity should be sufficient that vessel ingrowth may occur, but blood supply must be insufficient to maintain physiological calcium and/or phosphate ion concentrations.

One recent study compared two pore sizes of FDM scaffolds (0.6 mm and 1.2 mm), demonstrating in vivo that the larger pore size promotes enhanced bone formation [Freeman et al (2019) Biofabrication of multiscale bone extracellular matrix scaffolds for bone tissue engineering, European Cells and Materials, 38: 168-187.].

Scaffolds for regeneration of soft tissue and for cell delivery, formed by melt electrowetting (MEW), are described in WO2020210877, US2019106674 and US2019254959.

It is an object of the invention to overcome at least one of the above-referenced problems.

### SUMMARY OF THE INVENTION

The Applicant has discovered that melt electrowetting (MEW) may be employed to print fibrous bone regeneration scaffolds employing narrow diameter fibres (e.g. less than 40 μm in diameter) providing scaffolds with smaller pores (e.g. less than 800 μm). The resultant scaffolds have a higher specific surface area that conventional FDM printed bone regeneration scaffolds. This has been found to provide an environment in the MEW scaffold in which the cells are forced to branch out more in 3-D space due to the lower fibre diameter and lack of large curved surfaces on which to grow, with the result that the MEW scaffold displays enhanced healing compared with a conventional FDM scaffold in a rat femoral segmental defect model as shown by the volume of new bone formed after 12 weeks. MEW gives lower total level of vascularity, but similar amount of micro-vascularity (defined by vessels <20 μm in diameter. So, the comparably high number of micro-vessels in MEW, with less larger vessels, could be creating an appropriate environment for bone healing. The scaffolds of the invention are layered structures in which each layer comprise one or more fibres arranged in a lattice defining openings. The fibres in the lattice may be laid down in two or more orientations depending on the size of opening required. The openings of at least some layers are aligned to provide 3-D pores that extend through at least part of the scaffold. The layers may be arranged in groups to provide one or more columns of pores that are offset with respect to each other in an alternating fashion to present a composite pore that extends in a tortuous path through the scaffold (see FIG. 2B).

The Applicant also provides a scaffold having a varying architecture along the length of the scaffold and suitable for use in regeneration of defects in mixed tissue such as osteochondral defects. For example, the scaffold may have a first section formed by MEW that employs a fibre having a first diameter (e.g. 20 μm) and comprises a plurality of lattice layers with openings of a first size (e.g. 600 μm). This section of the scaffold is suitable for implanting in a bone part of an osteochondral defect. The scaffold may have a second section, formed by MEW that employs a fibre having a second diameter smaller than the first diameter (e.g., 10 μm) and comprises a plurality of lattice layers with openings of a second size smaller than the first size (e.g., 300 600 μm). This section of the scaffold is suitable for implanting in a chondral section of an osteochondral defect. The scaffold may have a third section formed by MEW that employs a fibre having a third diameter smaller or equal to the first diameter (e.g., 10 μm) and comprises a plurality of lattice layers with openings of a third size smaller than the second size (e.g., 50-150 μm). The third section may form a thin superficial tangential region that is introduced on top of the ‘chondral’ region to produce a tri-layered scaffold.

The Applicant has also discovered that the strength and structural rigidity of the scaffold of the invention may be improved by providing a shell support for the MEW printed scaffold, where the shell support is not formed by MEW but is 3-D printed using for example FDM printing with thicker fibres (e.g. fibre diameter of greater than 150 or 200 μm). An example is shown in Fig. X. The shell may take the form of a hollow sleeve dimensioned to receive the MEW printed scaffold. The sleeve may have an open top and/or open bottom. The sleeve may comprise circumferential struts.

In a first aspect, the invention provides a tissue regeneration scaffold comprising a first section comprising a plurality of first printed layers, each first printed layer comprising one or more printed fibres arranged in a lattice and defining a plurality of openings. The fibres typically have a diameter of less than 50 μm, 40 μm or 30 μm. The openings typically have a diameter of less than 1000 μm, 900 μm or 800 μm. At least some of the first printed layers are generally arranged such that the openings are aligned to define 3-D pores that extend at least partially through the first section. The one or more printed fibres are generally melt electrowritten.

In any embodiment, the scaffold (or the first section of the scaffold) has specific surface area of at least 100 mm²/mm³, 120 mm²/mm³, 150 mm²/mm³ or 160 mm²/mm³.

In any embodiment, the one or more fibres have a diameter of 10 to 30 μm or 15 to 25 μm.

In any embodiment, the openings have a width (e.g., fibre spacing) of 400 to 800 μm, 400 to 600 μm, 600 to 800 μm, 500 to 700 μm or 550 to 650 μm.

The openings generally have four sides and may be square or rectangular. The diameter of the openings is defined by the fibre lattice. Thus, if the fibre is laid down in two orientations in which the fibres arranged in the first orientation are parallel and equidistant to each other, and the fibres arranged in the second orientation are parallel and equidistant to each other, and orthogonal to the fibres in the first orientation, the openings will be square. The diameter of the opening is defined by the distance between fibres. The fibres in the second orientation are transverse to the fibres in the first orientation, and do not have to be orthogonal.

In any embodiment, the plurality of first printed layers comprise:
a first group of first printed layers comprising first 3-D pores; and
a second group of first printed layers comprising second 3-D pores,
   wherein the first 3-D pores are at least partially offset with respect to the second 3-D pores to define composite 3-D pores that extends at least partially (or fully) through the first section of the scaffold in a tortuous path.

The pores of the first group may be offset with respect to the pores of the second group in along an X-axis, Y-axis or both the X-axis and Y-axis. In the embodiment shown in FIG. 2, the pores of one group are offset with respect to the pores of the second group along both the X and the Y axis.

In any embodiment, the first group and second group of first printed layers each independently comprises 2-10 or 3 to 7 first printed layers.

In any embodiment, the first section of the scaffold comprises a plurality of first groups of first printed layers and a plurality of second groups of first printed layers in which the first and second groups of first printed layers are arranged in an alternating fashion. This arrangement provides an alternating offset pore structure providing a composite pore having a tortuous path through the scaffold.

In any embodiment, the first section of the scaffold has a height of 2 to 10 mm.

In any embodiment, the tissue regeneration scaffold comprises a second section disposed on top of the first section. The second section generally has a scaffold architecture that is different to the scaffold architecture of the first section, typically with smaller fibre diameter and smaller openings/voids. The second section may form a chondral section of the scaffold suitable for implantation into chondral defects.

In any embodiment, the second section of the scaffold comprises a plurality of second printed layers each comprising one or more fibres arranged in a lattice and defining a plurality of openings. The second printed layers are generally arranged such that the openings are aligned to define 3-D pores that extend through (typically fully through) the second section. The openings of the second printed layers are generally smaller than or equal to the openings of the first printed layers. The one or more printed fibres of the second printed layers generally have a diameter that is less than the one or more printed fibres of the first printed layers. The fibre or fibres of the second section are generally melt electrowritten.

In any embodiment, the second section of the scaffold may comprise or be coated with a growth factor, collagen matrix or a glycosaminoglycan (GAG).

In any embodiment, the one or more melt electrowritten fibres of the second printed layers have a diameter of 2 to 20 μm, 5 to 20 μm, 5 to 15 μm or 8 to 12 μm.

In any embodiment, the openings of the second printed layers have a diameter of 300 to 600 μm, 300 to 500 μm, 300 to 400 μm. 400 to 500 μm, or 500 to 600 μm.

In any embodiment, the second section of the scaffold has a height of 1 to 5 mm.

In any embodiment, the scaffold comprises a third section disposed on top of the section section, the third section comprising a plurality of third printed layers each comprising one or more melt electrowritten fibres arranged in a lattice, wherein:
the openings of the third printed layers are smaller than the openings of the second printed layers; and
the one or more printed fibres of the third printed layers have a diameter that is less than the one or more printed fibres of the first printed layers.

In any embodiment, the one or more melt electrowritten fibres of the third section have a diameter of 1 to 20 μm, 5 to 20 μm, 5 to 15 μm or 8 to 12 μm.

In any embodiment, the openings of the third section have a diameter of 50 to 150 μm.

In any embodiment, the third section of the scaffold has a height of 50 to 250 μm.

In any embodiment, the lattice of the third printed layers comprises the one or more printed fibres laid down in more than two orientations, for example three or four orientations, resulting in a denser lattice architecture.

In any embodiment, the scaffold comprises a fourth section (e.g., interface layer) disposed in between the first and second sections comprising a one or more fourth printed layers each comprising one or more melt electrowritten fibres arranged in a lattice, wherein:
the openings of the fourth printed layers are smaller than the openings of the second printed layers; and
the one or more printed fibres of the fourth printed layers have a diameter that is less than the one or more printed fibres of the first printed layers.

In any embodiment, the one or more melt electrowritten fibres of the fourth section have a diameter of 1 to 20 μm, 5 to 20 μm, 5 to 15 μm or 8 to 12 μm.

In any embodiment, the openings of the fourth section have a diameter of 50 to 150 μm.

In any embodiment, the fourth section of the scaffold has a height of 50 to 250 μm.

In any embodiment, the scaffold (or the first section of the scaffold) comprises a coating of hydroxyapatite. The hydroxyapatite may be needle shaped.

In any embodiment, a growth factor (for example bone morphogenic protein 2 (BMP2)) is embedded into the coating of hydroxyapatite.

In any embodiment, the first (e.g., osseous) section of the scaffold has a porosity of 90 to 99% or 95 to 99%.

In any embodiment, the second (e.g., chondral) section of the scaffold has a porosity of 90 to 99% or 95 to 99%.

In any embodiment, the third (e.g., superficial) section of the scaffold has a porosity of 80 to 90% or 80 to 85%.

In another aspect, the invention provides a tissue regeneration scaffold device having a core-shell structure, in which the core comprises a tissue regeneration scaffold according to the invention and the shell is hollow and dimensioned to receive the core. The shell generally comprises a sleeve structure and/or a 3-D printed exoskeleton that embraces at least the first section of the tissue regeneration scaffold. The shell is generally a FDM printed scaffold. The fibres or struts of the shell generally have a fibre diameter of at least 100 μm, 150 μm or 200 μm. The shell generally surrounds the sides of the scaffold leaving the top and/or bottom uncovered.

In another aspect, the invention provides a tissue regeneration scaffold comprising a melt electrowritten (MEW) scaffold core contained within a printed scaffold shell. The printed scaffold shell may be FDM printed. The tissue regeneration scaffold may be a bone or osteochondral regeneration scaffold.

In any embodiment, the MEW scaffold core comprises a first section comprising a plurality of first printed layers, each first printed layer comprising one or more printed fibres arranged in a lattice and defining a plurality of openings. The fibres typically have a diameter of less than 50 μm, 40 μm or 30 μm. The openings typically have a diameter of less than 1000 μm, 900 μm or 800 μm. At least some of the first printed layers are generally arranged such that the openings are aligned to define 3-D pores that extend at least partially through the first section. The one or more printed fibres are generally melt electrowritten.

In any embodiment, the MEW scaffold core has a specific surface area of at least 100 mm²/mm³, 120 mm²/mm³, 150 mm²/mm³ or 160 mm²/mm³.

In any embodiment, the one or more fibres of the MEW scaffold core have a diameter of 10 to 30 μm or 15 to 25 μm.

In any embodiment, the openings MEW scaffold core have a diameter of 400 to 800 μm, 400 to 600 μm, 600 to 800 μm, 500 to 700 μm or 550 to 650 μm.

The printed scaffold shell may comprise (or be formed of) a printed fibre having a diameter of greater than 100 μm, 150 μm or 200 μm.

In another aspect, the invention provides a method of manufacturing a first section of a tissue regeneration scaffold of the invention, comprising:
melt electrowriting a plurality of first printed layers on top of each other in an iterative fashion, in which each first printed layer comprises one or more printed fibres (typically having a diameter of less than 40 μm) arranged in a lattice and defining a plurality of openings (typically having a diameter of less than 800 μm), and in which the openings of at least some of the first printed layers overlap to provide a plurality of pores that extend at least partially through the scaffold.

In any embodiment, the melt electrowriting step comprises the steps of:
(a) melt electrowriting a first group of first printed layers to provide a first scaffold part comprising first pores extending through the first scaffold part;
(b) melt electrowriting a second group of first printed layers to provide a second scaffold part comprising second pores extending through the second scaffold part that are offset with respect to the first pores; and
(c) optionally repeating steps (a) and (b) to provide a multi part scaffold with composite pores that extend through the scaffold in a tortuous path.

In any embodiment, the method comprises a step of coating the scaffold in hydroxyapatite.

In any embodiment, the method comprises a step of embedding a biological growth factor in the hydroxyapatite coating.

In any embodiment, the tissue regeneration scaffold comprises a second section disposed on top of the first section, in which the method comprises a step of forming the section section by melt electrowriting. The second section generally has a scaffold architecture that is different to the scaffold architecture of the first section, typically with smaller fibre diameter and smaller openings/voids. The second section may form a chondral section of the scaffold suitable for implantation into chondral defects.

In any embodiment, the method comprises melt electrowriting a plurality of second printed layers each comprising one or more fibres arranged in a lattice and defining a plurality of openings. The second printed layers are generally arranged such that the openings are aligned to define 3-D pores that extend through (typically fully through) the second section. The openings of the second printed layers are generally smaller than the openings of the first printed layers. The one or more printed fibres of the second printed layers generally have a diameter that is less than the one or more printed fibres of the first printed layers. The fibre or fibres of the second section are generally melt electrowritten.

In any embodiment, the method comprises melt electrowriting one or more fibres of the second printed layers having a diameter of 2 to 20 μm, 5 to 20 μm, 5 to 15 μm or 8 to 12 μm.

In any embodiment, the method comprises melt electrowriting one or more fibres of the second printed layers such that the openings of the second printed layers have a diameter of 300 to 600 μm, 300 to 500 μm, 300 to 400 μm. 400 to 500 μm, or 500 to 600 μm.

In any embodiment, the second section of the scaffold has a height of 1 to 5 mm.

In any embodiment, the method comprises melt electrowriting a plurality of third printed layers each comprising one or more melt electrowritten fibres arranged in a lattice, wherein:
the openings of the third printed layers are smaller than the openings of the second printed layers; and
the one or more printed fibres of the third printed layers have a diameter that is less than the one or more printed fibres of the first printed layers.

In any embodiment, the one or more melt electrowritten fibres of the third section have a diameter of 1 to 20 μm, 5 to 20 μm, 5 to 15 μm or 8 to 12 μm.

In any embodiment, the openings of the third section have a diameter of 50 to 150 μm.

In any embodiment, the third section of the scaffold has a height of 50 to 250 μm.

In any embodiment, the lattice of the third printed layers comprises the one or more printed fibres laid down in more than two orientations, for example three or four orientations, resulting in a denser lattice architecture.

In another aspect, the invention provides a method of manufacturing a tissue regeneration scaffold device of the invention, comprising the steps of:
manufacturing a tissue regeneration scaffold according to a method of the invention;
printing a 3-D exoskeleton for the tissue regeneration scaffold; and
placing the tissue regeneration scaffold inside the 3-D exoskeleton.

In any embodiment, the 3-D exoskeleton is printed by FDM printing.

In another aspect, the invention provides a method of treating a tissue defect such as a bone or osteochondral defect in a mammal, comprising the steps of providing a tissue regeneration scaffold or tissue regeneration scaffold device of the invention, optionally shaping the scaffold or device to fit into the defect, optionally soaking the scaffold, either before or after the optional shaping, in a solution of biological material, and inserting the scaffold into the defect. Optionally, the biological material can be incorporated into the scaffolds during the fabrication process.

Other aspects and preferred embodiments of the invention are defined and described in the other claims set out below.

### BRIEF DESCRIPTION OF THE FIGURES

FIG. 1A: Perspective view of a MEW printed bone regeneration scaffold of the invention in which the openings of the layers are aligned to provide “open pores”.

FIG. 1B: Side elevational view of the MEW printed bone regeneration scaffold of FIG. 1A

FIG. 1C: Perspective view of a MEW printed layer forming part of the scaffold of FIG. 1A.

FIG. 1D: Plan view of the MEW printed layer of FIG. 1C.

FIG. 2A: Perspective view of a MEW printed bone regeneration scaffold of the invention having groups of layers configured to provide composite pores that extend through the scaffold in a tortuous path (“Offset pores”).

FIG. 2B: Side elevational view of the MEW printed bone regeneration scaffold of FIG. 2A showing the offset pores

FIG. 2C: Perspective view of a MEW printed layer of one group of layers and a second MEW printed layer of a second group of layers, forming part of the scaffold of FIG. 2A.

FIG. 2D: Plan view of the MEW printed layer of FIG. 2C.

FIG. 3A: Perspective view of an osteochondral regeneration scaffold of the invention having a first (osseous) section with first pores and a second (chondral) section on top of the first section having smaller pores, and an interface region with a narrow pore size disposed between the first and second section

FIG. 3B: Side elevational view of the osteochondral scaffold of FIG. 3A.

FIG. 3C: Exploded view of the osteochondral scaffold of FIG. 3A.

FIG. 4A: Simplified representation of perspective view of a FDM printed shell forming part of a core-shell bone regeneration scaffold device of the invention

FIG. 4B: Perspective view of a MEW printed scaffold forming a core of a core-shell bone regeneration scaffold device of the invention

FIG. 4C: Simplified representation of perspective view of a core-shell scaffold device of the invention comprising the MEW scaffold of FIG. 4B contained within the FDM printed supporting shell of FIG. 4A.

FIG. 4D: Perspective view of toolpath and layers used to print the FDM shell

FIG. 4E: Another perspective view of toolpath and layers used to print the FDM shell

FIG. 4F: Stereomicroscope image of final assembled core-shell scaffold

FIG. 4G: Scanning electron microscope image of final assembled core-shell scaffold

FIG. 5A: Perspective view of a scaffold device according to the invention having a core comprising a tri-layer osteochondral scaffold (that is similar to the osteochondral scaffold of FIG. 3) and a FDM printed shell in the form of a hollow sleeve dimensioned to receive and support the osteochondral scaffold

FIG. 5B: Exploded view of the scaffold device of FIG. 5A.

FIG. 6A: is an image of the scaffold device looking in the direction of arrow X in FIG. 5B

FIG. 6B: is an image of the scaffold of the invention looking in the direction of arrow Y in FIG. 5B.

FIG. 7A: is immunofluorescent images of FDM and MEW scaffolds after 24 hours demonstrating comparable seeding efficiency and cell attachment between them.

FIG. 7B: is DNA content in scaffolds after 1 day and 21 days in culture, demonstrating enhanced proliferation in the MEW scaffold after 21 days.

FIG. 7C: is ALP activity in scaffolds after 1 day and 21 days in culture, demonstrating enhanced ALP activity in the MEW scaffold after 21 days.

FIG. 7D: is calcium content in scaffolds after 1 day and 21 days in culture, demonstrating enhanced mineralisation in the MEW scaffold after 21 days.

FIG. 7E: is scanning electron microscopy images after 21 days in culture. Dense matrix is produced by cells throughout the depth of the pores in the MEW group.

FIG. 8A: are 3D models from reconstructed in vivo μCT scan slices 12 weeks after implantation of scaffolds. Healing in the MEW scaffold group is characterised by a round bone front, in contrast to discrete spicules being formed throughout the pores in the FDM group.

FIG. 8B: is quantification of bone volume considering only the 5 mm defect between the bone ends. Bone volume is significantly enhanced after 12 weeks in the MEW scaffold group demonstrating the enhanced healing which it provides.
*=statistical significance between groups using mixed-effects analysis and Tukey's multiple comparisons test (*=p<0.05). #=statistical significance within groups between time-points using mixed-effects analysis and Bonferroni's multiple comparison test. (###=p<0.001).

FIG. 9A: is Goldner's Trichrome staining of the Empty scaffold group.

FIG. 9B: is Goldner's Trichrome staining of the FDM scaffold group demonstrating both large and small vessels.

FIG. 9C: is Goldner's Trichrome staining of the MEW scaffold group showing predominantly smaller vessels.

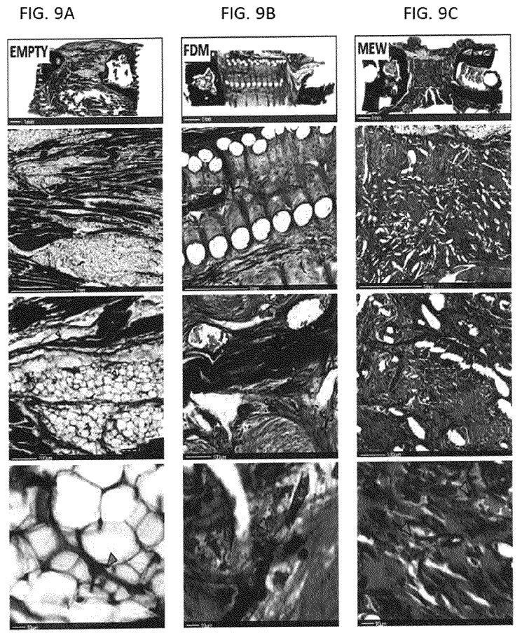

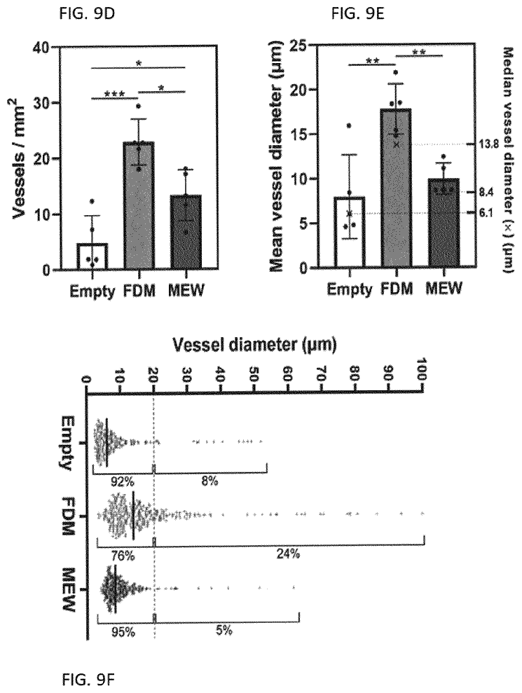

FIG. 9D-9F: is quantification of vessel density in each group (FIG. 9D), mean and median vessel diameter (FIG. 9E), and spread of data for vessel diameter (FIG. 9F), demonstrating a similar quantity of smaller vessels in both FDM and MEW groups, with a lesser number of larger vessels in the MEW group. There is evidence in the literature of a more complex relationship between vascularisation and bone formation [1-3], where restricted or more controlled vascularisation and oxygen levels may indeed provide beneficial.
*=statistical significance between groups using unpaired t test (**=p<0.01, ***=p<0.001).

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, age, poisoning or nutritional deficiencies.

As used herein, the term “treatment” or “treating” refers to an intervention (e.g. the administration of an agent to a subject) which cures, ameliorates or lessens the symptoms of a disease or removes (or lessens the impact of) its cause(s) (for example, the reduction in accumulation of pathological levels of lysosomal enzymes). In this case, the term is used synonymously with the term “therapy”.

Additionally, the terms “treatment” or “treating” refers to an intervention (e.g. the administration of an agent to a subject) which prevents or delays the onset or progression of a disease or reduces (or eradicates) its incidence within a treated population. In this case, the term treatment is used synonymously with the term “prophylaxis”.

As used herein, an effective amount or a therapeutically effective amount of an agent defines an amount that can be administered to a subject without excessive toxicity, irritation, allergic response, or other problem or complication, commensurate with a reasonable benefit/risk ratio, but one that is sufficient to provide the desired effect, e.g. the treatment or prophylaxis manifested by a permanent or temporary improvement in the subject's condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure. Improvement may be observed in biological/molecular markers, clinical or observational improvements. In a preferred embodiment, the methods of the invention are applicable to humans, large racing animals (horses, camels, dogs), and domestic companion animals (cats and dogs).

In the context of treatment and effective amounts as defined above, the term subject (which is to be read to include “individual”, “animal”, “patient” or “mammal” where context permits) defines any subject, particularly a mammalian subject, for whom treatment is indicated. Mammalian subjects include, but are not limited to, humans, domestic animals, farm animals, zoo animals, sport animals, pet animals such as dogs, cats, guinea pigs, rabbits, rats, mice, horses, camels, bison, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; and rodents such as mice, rats, hamsters and guinea pigs. In preferred embodiments, the subject is a human. As used herein, the term “equine” refers to mammals of the family Equidae, which includes horses, donkeys, asses, kiang and zebra.

“Tissue regeneration scaffold” refers to a porous structure suitable for implantation in the body for the purpose of regeneration and repair of tissue, especially hard tissue like bone and cartilage. The scaffold is suitable for treatment of critical size bone and osteochondral defects. The scaffold is formed from MEW printed layers that are printed on each other in an iterative manner to build-up the scaffold. The scaffold may be fully or partially coated with a mineral such as hydroxyapatite. When the scaffold has an osseous section and a chondral section, the osseous section exclusively may be coated with mineral. A biological growth factor such as BMP-2 may be embedded into the mineral coat. A biological growth factor, collagen matrix or glycosaminoglycan may be coated on to the chondral section of the scaffold. The scaffold may be printed to dimensionally match a defect. The scaffold may be printed and then shaped post-printing to a suitable size. The scaffold may be cylindrical. The scaffold may have a height of 1 to 10 mm. The scaffold may have a diameter of 0.5 to 5 mm. The scaffold may be seeded with cells such as for example chondrocytes, osteoblasts or mesenchymal stem cells. In any embodiment, the scaffold is biodegradable.

“Melt electrowritten” or “MEW” refers to is a high-resolution additive manufacturing method with the capability to produce fibers in the micro-and nanometer range using a similar configuration to melt electrospinning, except using different parameters and with a moving collector that facilitates direct-writing. It is described in Kade & Dalton (2020) Polymers for Melt Electrowriting, Advanced Healthcare Materials.

https://doi.org/10.1002/adhm.202001232

A custom MEW printer designed and built in-house was used to fabricate MEW scaffolds. This printer has a 3D positioning system comprising of XSlide linear slides (Velmex, USA) driven by NEMA 23 stepper motors (Arcus, USA). The print head is comprised of a custom polyether ether ketone (PEEK) housing assembly containing 2 ceramic heaters with proportional-integral-derivative (PID) control. Air pressure for extrusion is controlled by a proportional pressure regulator with PID control of pneumatic pressure (Festo, Ireland). Voltage is applied to the syringe needle via a high voltage power supply (Heinzinger, Germany), while the print plate is grounded. All components (motorised linear slides, heaters, pressure controller, high voltage power supply, safety relays/contactors and interlocks) are centrally controlled via a custom control box with an 8-axis motion controller (Trio Motion, UK). PCL (CAPA 6500D) was loaded to 3 ml syringes with 22 G needles (Nordson EFD) and placed in the MEW print head. PCL was extruded at a pressure of 0.8 Bar and temperatures of 85° C. and 90° C. at the barrel and needle heaters respectively within the print head. An initial voltage of 7 kV was applied with a needle-plate height of 8 mm. Scaffolds were printed with a translation speed of 6 mm/s with dimensions 60×60 mm for the first 2 layers to form a large brim for enhanced print plate adhesion. The remainder of scaffolds were printed with dimensions 48×48 mm with a total number of 220 layers including the brim (with each layer comprising fibres in the X and Y direction). Voltage and print height were incrementally increased at each layer of the print up to a final scaffold height of either 2 mm or 4.8 mm, with a layer print head increase of 22.7 μm and a layer voltage increase of 0.0129 kV. Scaffolds were punched to a diameter of 4 mm using biopsy punches.

“Printed layer” as used herein refers to a layer of the scaffold of the invention. The scaffold comprises a plurality of printed layers printed on top of each other. Each layer is generally provided in the form of a lattice comprising at least a first part with fibres extending in a first orientation and a second part with fibres extending in a second orientation transversely to the fibres of the first part and forming openings defined by the intersection of the fibres of the first and second parts. The fibres generally intersect at right angles but this is not essential. The fibres of each part are generally equally spaced apart but this is also not essential. When the fibres are all equally spaced apart and intersect at right angles, the openings formed are square. The lattice may also include a third part comprising fibres. The lattice may also include strengthening fibres that have a diameter greater than the fibres making up the bulk of the lattice. The scaffold may have different sections, in which the layers of each section may differ from each other in terms of fibre diameter and/or opening diameter.

“Fibre” as used herein refers to a fibre suitable for 3-D printing, examples include Synthetic polymers such as poly(-caprolactone) (PCL), poly(lactic acid) (PLA), poly(glycolic acid) (PGA), poly(lactic-co-glycolic acid) (PLGA). In any embodiment, the PLGA is CAPA 6500D.

“Hydroxyapatite” or “HA”. HA is generally coated on the scaffold using a wet precipitation method. Calcium and Phosphorus based solutions are combined with the scaffold immersed within to result in a thin layer (approx. 0.55 μm). The HA generally has a needle structure. These needle-structured mineral units have an aggregate size which is ≈100 nm in length with a thickness of 37.04 nm as imaged by SEM and are seen to form occasional aggregates which range in diameter from 150 to 500 nm. TEM imaging reveals a smaller particle unit with length 58.19 nm and thickness 10.42 nm. Full characterisation of this coating method is described in Eichholz et al (2020) Development of a New Bone-Mimetic Surface Treatment Platform: Nanoneedle Hydroxyapatite (nnHA) Coating, Advanced Healthcare Materials.

https://doi.org/10.1002/adhm.202001102

“Growth factor” More recent advances in bone or cartilage tissue engineering involve the use of scaffolds as growth factor or gene carrier systems. There are a number of essential growth factors providing regulatory effects on chondrocytes or stem cells involved in chondrocyte maturation and cartilage formation. These include the TGF-β superfamily, IFG, FGF, BMP, PDGF and EGF (Lee S H, Shin H; 2007). Miljkovic et al. (Miljkovic et al.; 2008) report on the successful delivery of BMP-4 for the treatment of cartilage defects. Thus, in one embodiment of the invention, the process includes an additional step of soaking the multi-layer collagen-composite scaffold in a solution of biological material to allow biological material infiltrate into the porous architecture of the scaffold. Suitably, the biological material is selected from the groups of: cells; and biological growth factors. Typically, the biological growth factors are selected from the group consisting of one or more of the TGF-β superfamily, (IFG, FGF, BMP, PDGF, EGF) or cannabinoids. These growth factors can also be included during the production process as opposed to post-fabrication soaking of the scaffolds. Additionally, these scaffolds are ideally suited for use as delivery mechanisms for gene therapy delivery, either through viral or non-viral delivery vectors. The idea of a gene delivery vector contained within a biodegradable scaffold, although not new, is a recent development in the field of regenerative medicine and the system has been coined as a ‘gene activated matrix’ (GAM). Gene therapy can be a valuable tool to avoid the limitations of local delivery of growth factors, including short half-life, large dose requirement, high cost, need for repeated applications, and poor distribution.

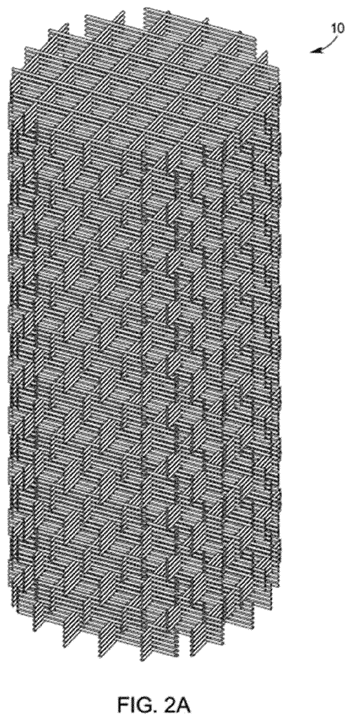

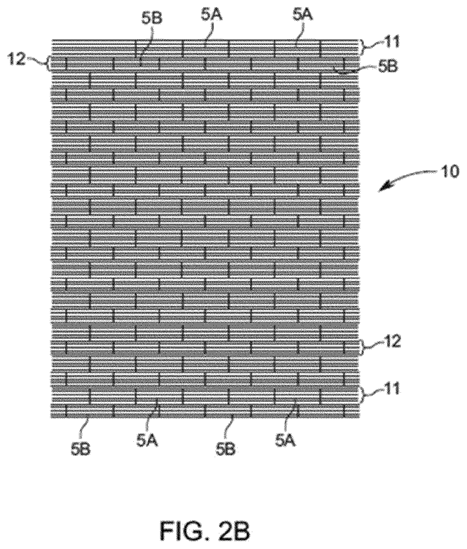

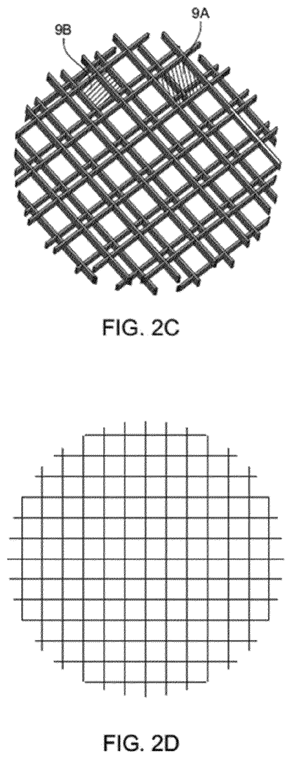

“Tortuous path” refers to the path defined by a composite pore in the scaffold may up by a column of pores that partially overlap. This achieved by printing the layers of the scaffold in groups, where a first group of layers have aligned openings that form a first pore that extends through the group of layers, and a second group of layers have aligned openings that form a second pore that extends through the second group of layers, where the first and second pores only partially overlap. The pores of the respective groups may be offset along an X-axis, Y-axis, or both. This illustrated in FIG. 2

“Core-shell”. The invention provides a tissue regeneration scaffold device comprising a core scaffold contained within a shell scaffold. The core scaffold is provided by the tissue regeneration scaffold of the invention which is formed by MEW. The shell is 3-D printed using a different printing technique such as FDM. The shell is printed using fibres having a bigger diameter that the fibres of the core (e.g. greater than 100, 150 or 200 μm). The shell may take the form of a hollow sleeve dimensioned to receive the core scaffold.

### Exemplification

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

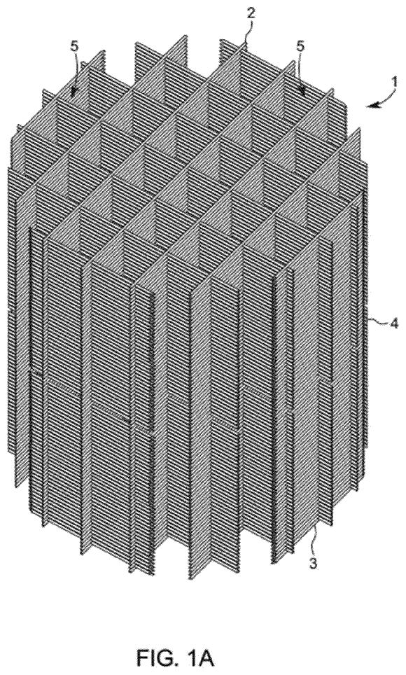

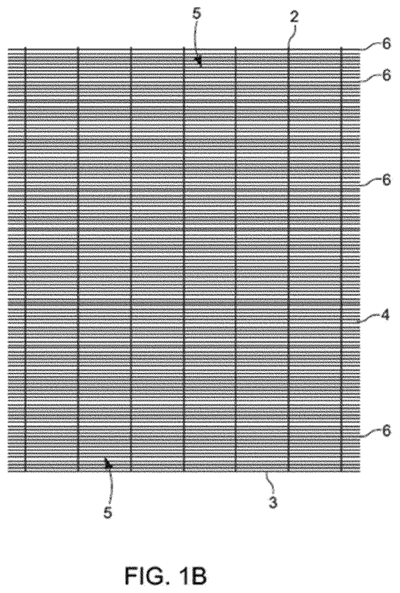

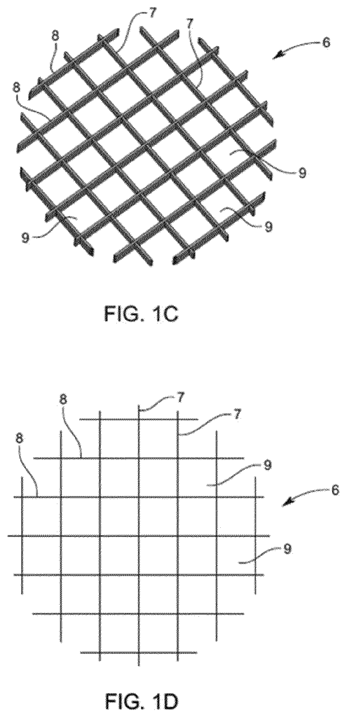

Referring to the drawings and initially to FIG. 1, a bone regeneration scaffold according to the invention is illustrated, indicated generally by the reference numeral 1. The scaffold 1 has a generally cylindrical shape with a top 2, bottom 3 and sidewall 4, and a plurality of cubic pores 5 that extend through the scaffold form the top 2 to the bottom. These pores are referred to herein as open pores. The pores have a pore width of about 600 μm. The scaffold has a width (diameter) of about 4 mm and a height of about 4.8 mm.

The scaffold is made up of multiple layers formed by MEW printing that are printed on top of each other to form the 3D scaffold. In the embodiment shown, the scaffold has about 220 layers. A single MEW printed layer 6 is shown in FIGS. 1C and 1D and comprises a first part comprising first PLGA fibres 7 having a diameter of 20 μm extending in a first orientation and a second part comprising second PLGA fibres 8 having a diameter of 20 μm extending orthogonally to the first fibres 7 and intersecting to form a lattice with square openings 9 having a width of 600 μm. In the scaffold of this embodiment of the invention, the layers 6 are printed with the openings 9 in register with each other so that the openings forms the cubic pores 5 that extend through the scaffold from the top 2 to the bottom 3. These open pores are shown in FIG. 1B. This scaffold is suitable for use as a bone regeneration scaffold and may be shaped post-printing or printed to the dimensioned required to fit into a bone defect.

Referring to FIG. 2, an alternative scaffold of the invention is described in which parts described with reference to the previous embodiment are assigned the same reference numerals. In this embodiment, the scaffold, indicated generally by the reference numeral 10, has groups of printed layers including first groups 11 and second groups 12 which are printed in an alternating fashion as indicated in FIG. 2B. Each first group 11 of printed layers has five layers formed as described with reference to FIG. 1, in which the openings 9A (shown cross-hatched in FIG. 2C) are in register to form pores 5A. Each second group 12 of printed layers has five layers formed as described with reference to FIG. 1, in which the openings 9B (shown with different cross-hatching) are in register with each other to form pores 5B, but out of register in both the X and Y direction) with the openings of the first group 11 of layers such that the pores 5B are laterally offset with respect to the pores 5A of the first group and thereby define a composite pore that extends through the scaffold along a tortuous path. FIGS. 2C and 2D illustrate a printed layer of a first group 11 on top of a printed layer of a second group, showing how the lattice network and respective openings of the printed layers form the first and second groups are not in register.

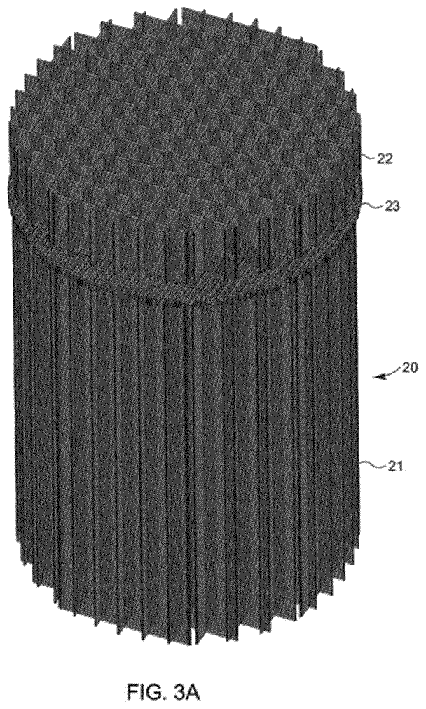

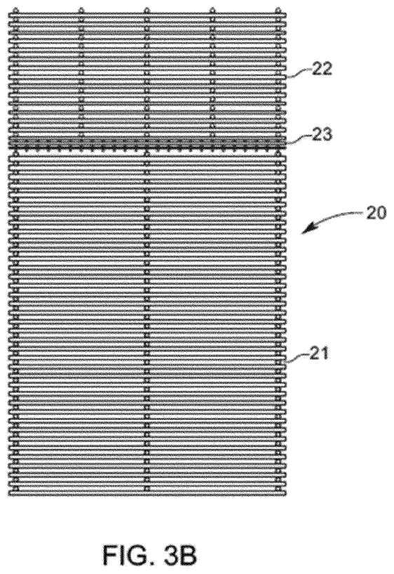

Referring to FIG. 3, an osteochondral regeneration scaffold of the invention, indicated generally by the reference numeral 20 is described in which parts described with reference to the previous embodiments are assigned the same reference numerals. The osteochondral scaffold 20 has a first (osseous) section 21, and a second (chondral) section 22. The first section 21 is formed by MEW printing according to the embodiment described with reference to FIG. 1 with open pores having a width (fibre spacing) of about 600 μm, and a fibre diameter of about 20 μm. The second section 22 is also formed by MEW and is printed in layers as described above but with a smaller fibre diameter of about 10 μm and with openings having a width of about 400 μm that are vertically aligned to form open pores with a pore width of about 400 m. The second section has the same diameter as the first section, and a height of about 25% of the height of the first section and comprises about 50 MEW printed layers. An interface layer 23 is MEW printed on top of the first section 21 and comprises a dense lattice network with interfibre spacings of 50-100 μm.

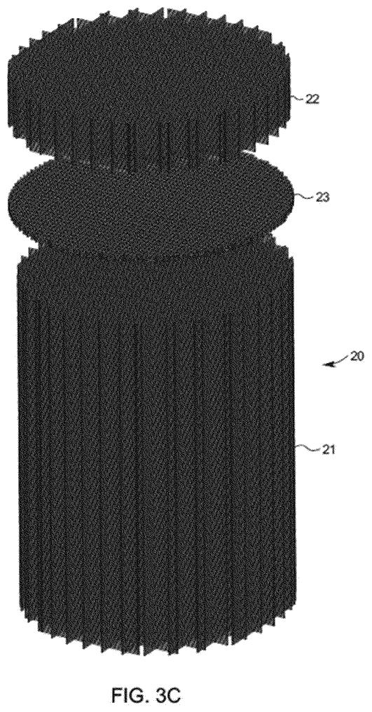

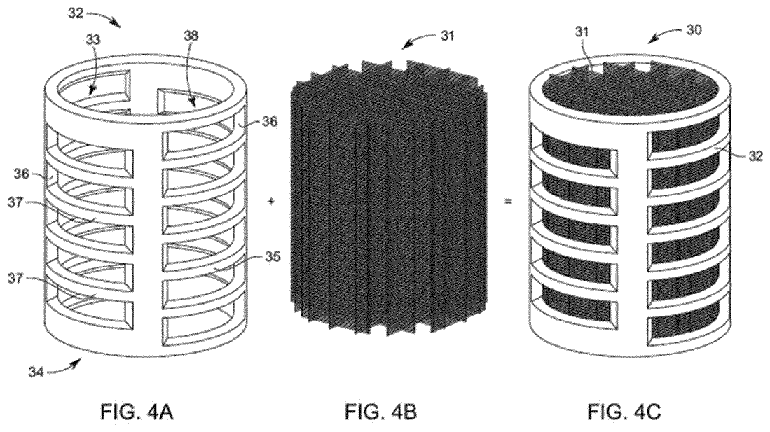

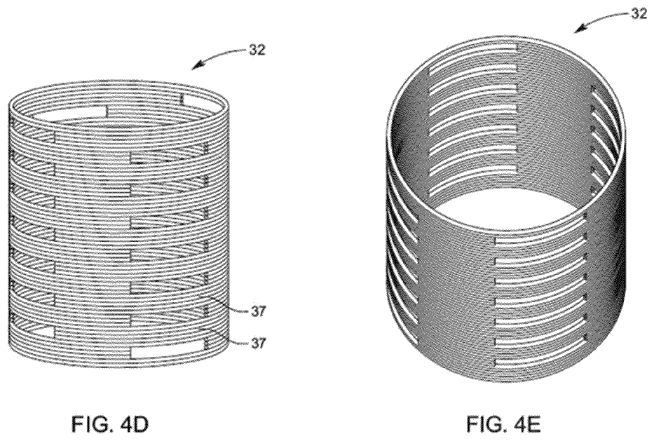

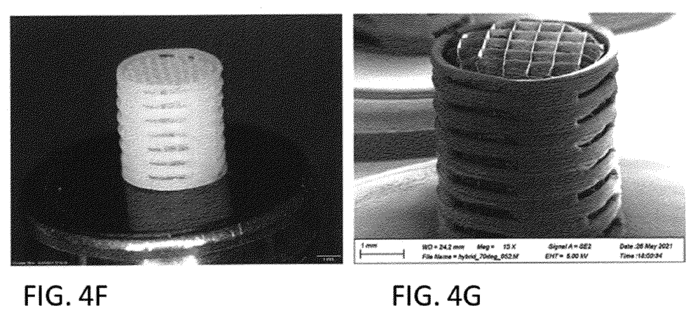

FIG. 4 illustrates a bone regeneration scaffold according to the invention, indicated generally by the reference numeral 30, in which parts described with reference to the previous embodiments are assigned the same reference numerals. The embodiments of the scaffold of the invention is suitable for use in bone repair/regeneration and has a core-shell structure formed using two different forms of 3-D printing. The core 31 is formed by MEW printing and comprises the scaffold 1 described with reference to FIG. 1. The shell comprises a FDM printed sleeve 32 having an open top 33, open bottom 34 and sidewall 35 defined by axial struts 36 (having a thickness of about 1 mm) and circumferential ribs 37 (having a thickness of about 500 μm) and hollow interior 38. The shell is FDM printed with a fibre having a diameter of about 200 μm and the struts and ribs are created by stacking multiple fibres on top of each other during printing. The hollow interior 38 of the shell is dimensioned to receive the scaffold 1 in a snug nested arrangement with the MEW fibres sticking out and engaging in pores of the FDM sleeve. The shell has a height that is the same as the height of the scaffold 1.

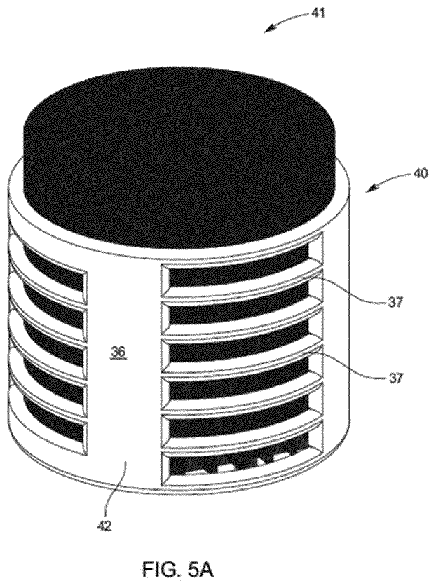

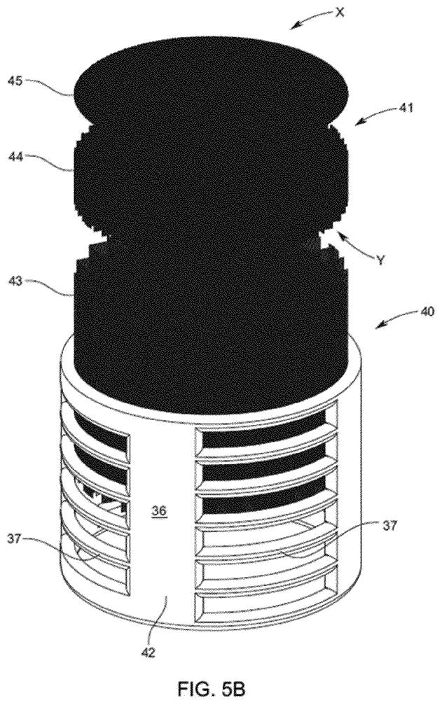

Referring to FIG. 5, an osteochondral regeneration scaffold device of the invention, indicated generally by the reference numeral 40 is described in which parts described with reference to the previous embodiments are assigned the same reference numerals. The device 40 has a core 41 and a shell 42. The core 41 comprises an osteochondral scaffold having a first (osseous) section 43, and a second (chondral) section 44 and a tangential superficial layer 45 on top of the ‘chondral’ region to produce the tri-layered scaffold core 41. The fiber-fiber spacing in this superficial layer will be lower than other regions of the scaffold (50-100 μm), in order to define the superficial tangential zone in the chondral region. Fibres will be laid down in four orientations in this layer to further reduce pore size and enhance fibre density (see appendix 3). The first section 43 and second section 44 are as described with reference to FIG. 3.

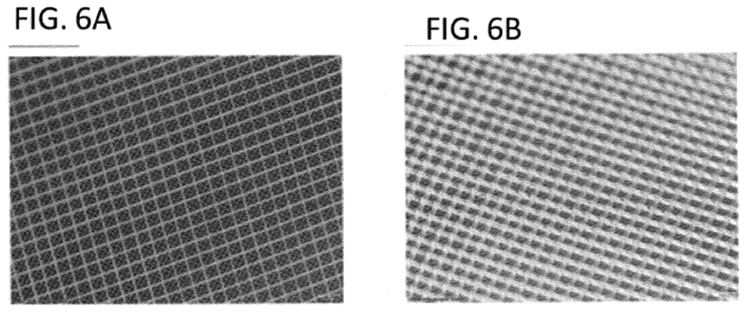

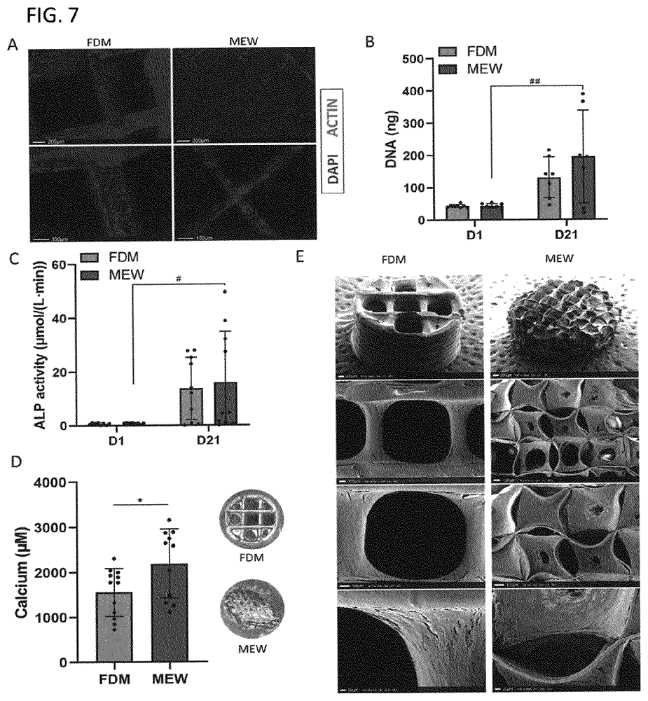

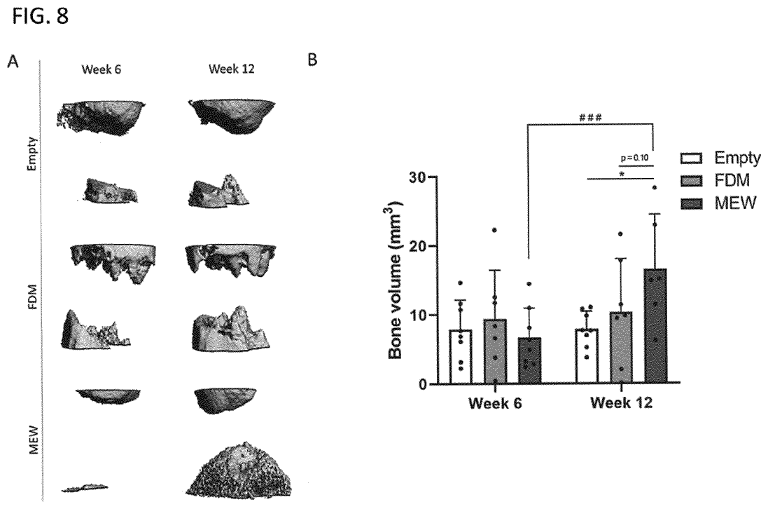

FIG. 6A is an image of the scaffold device looking in the direction of arrow X in FIG. 5B and FIG. 6B is an image of the scaffold of the invention looking in the direction of arrow Y in FIG. 5B.

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. A tissue regeneration scaffold comprising a first section comprising a plurality of first printed layers,
each first printed layer comprising one or more printed fibres having a diameter of less than 40 μm arranged in a lattice and defining a plurality of openings having a diameter of less than 800 μm, wherein:
at least some of the first printed layers are arranged such that the openings are aligned to define 3-D pores that extend at least partially through the first section; and
the one or more printed fibres are melt-electrowritten.

2. The tissue regeneration scaffold according to claim 1, having a specific surface area of at least 150 mm²/mm³.

3. The tissue regeneration scaffold according to claim 1, in which the one or more fibres have a diameter of 10 to 30 μm and the openings have a diameter of 400 to 800 μm.

4. The tissue regeneration scaffold according to claim 1, in which the plurality of first printed layers comprise:
a first group of first printed layers comprising a first set of 3-D pores; and
a second group of first printed layers comprising a second set of 3-D pores,
wherein the first set of 3-D pores are offset with respect to the second set of 3-D pores to define a set of composite 3-D pores having a tortuous path.

5. The tissue regeneration scaffold according to claim 4, in which the first group and second group of first printed layers each independently comprises 2-10 first printed layers.

6. The tissue regeneration scaffold according to claim 4, including a plurality of first groups of first printed layers and a plurality of second groups of first printed layers in which the first and second groups of first printed layers are arranged in an alternating fashion.

7. The tissue regeneration scaffold according to claim 1, further comprising a coating of hydroxyapatite.

8. The tissue regeneration scaffold according to claim 7, further comprising bone morphogenic protein 2 (BMP2) embedded into the coating of hydroxyapatite.

9. The tissue regeneration scaffold according to claim 1, having a second section disposed on top of the first section, the second section comprising a plurality of second printed layers each comprising one or more melt electrowritten fibres arranged in a lattice and defining a plurality of openings,
wherein the second printed layers are arranged such that the openings are aligned to define 3-D pores that extend through the second section,
wherein:
the openings of the second printed layers are smaller than the openings of the first printed layers; and
the one or more printed fibres of the second printed layers have a diameter that is less than the one or more printed fibres of the first printed layers.

10. The tissue regeneration scaffold according to claim 9, in which the one or more melt electrowritten fibres of the second printed layers have a diameter of 5 to 15 μm and the openings have a diameter of less than 300 to 600 μm.

11. The tissue regeneration scaffold according to claim 9, having a third section disposed on top of the second section, the third section comprising a plurality of third printed layers each comprising one or more melt electrowritten fibres arranged in a lattice, wherein:
the openings of the third printed layers are smaller than or equal to the openings of the second printed layers; and
the one or more printed fibres of the third printed layers have a diameter that is less than the one or more printed fibres of the first printed layers.

12. The tissue regeneration scaffold according to claim 11, in which the one or more melt electrowritten fibres of the third printed layers have a diameter of 5 to 15 μm and the openings have a diameter of less than 50 to 150 μm.

13. The tissue regeneration scaffold according to claim 11, in which the first section of the scaffold has a height of 2 to 10 mm;
the second section of the scaffold has a height of 1 to 5 mm; and
the third section of the scaffold has a height of 50 to 250 μm.

14. A tissue regeneration scaffold device having a core-shell structure, in which the core comprises the tissue regeneration scaffold according to claim 1 and the shell comprises a 3-D printed body that embraces the first section of the tissue regeneration scaffold.

15. The tissue regeneration scaffold device according to claim 14, in which the shell is a hollow cylindrical sleeve that is formed by FDM printing and the core is nested within the hollow cylindrical sleeve.
